(19) United States
(12) Patent Application Publication
(10) Pub. No.: US 2013/0172554 A1
(43) Pub. Date: Jul. 4, 2013

(54) PROCESSES FOR THE PREPARATION OF 4-MORPHOLIN-3-ONE

(71) Applicants: Dodda Mohan Rao, Hyderabad (IN); Pingili Krishna Reddy, Hyderabad (IN); Ambati Anna Reddy, Karimnagar (IN); Buthukuri Venkat Reddy, Hyderabad (IN)

(72) Inventors: Dodda Mohan Rao, Hyderabad (IN); Pingili Krishna Reddy, Hyderabad (IN); Ambati Anna Reddy, Karimnagar (IN); Buthukuri Venkat Reddy, Hyderabad (IN)

(73) Assignee: SYMED LABS LIMITED, Hyderabad, Andhra Pradesh (IN)

(21) Appl. No.: 13/821,182

(22) PCT Filed: Aug. 1, 2011
(86) PCT No.: PCT/IN11/00504

§ 371 Date: Mar. 6, 2013

(30) Foreign Application Priority Data
Sep. 7, 2010 (IN) 2609/CHE/2010

(51) Int. Cl. C07D 413/10; C07D 265/32; C07D 413/14

(52) U.S. Cl. CPC C07D 413/10; C07D 413/14; C07D 265/32
USPC 544/137; 544/166

(57) ABSTRACT

The present invention provides processes for the preparation of 4-{4-[5(S)-(aminomethyl)-2-oxo-1,3-oxazolidin-3-yl]phenyl}morpholin-3-one which are simple, eco-friendly, cost-effective, reproducible, robust and are well amenable on industrial scale.

### PRIORITY

This application claims the benefit of Indian Provisional Application with no.2609/CHE/2010 filed on 7 Sep. 2010 the contents of each of which are incorporated by reference herein.

### BACKGROUND OF THE INVENTION

1. Technical Field

The present invention relates to processes for the preparation of 4-{4-[5(S)-(aminomethyl)-2-oxo-1,3-oxazolidin-3-yl]phenyl}morpholin-3-one of formula (II) a key intermediate in the synthesis of rivaroxaban (I).

The intermediate compound of formula II is represented by

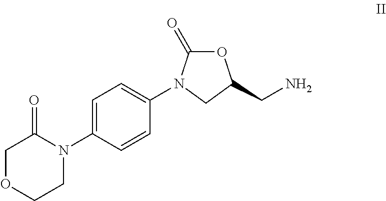

2. Description of the Related Art

Rivaroxaban is a novel anticoagulant used for the prevention of venous thromboembolism in adult patients undergoing elective hip or knee replacement surgery and is approved in US and Europe. Rivaroxaban is structurally related to the antibacterial compound Linezolid (Zyvox) is enantiomerically pure. Rivaroxaban is available in the market under the brand name Xarelto® as 10 mg tablets in Europe. Rivaroxaban is chemically described as 5-chloro-N-({(5S)-2-oxo-3-[4-(3-oxo-4-morpholinyl)phenyl]-1,3-oxazolidin-5-yl}methyl)-2-thiophene-carboxamide (herein after referred as rivaroxaban) and is represented by the structural formula I shown below:

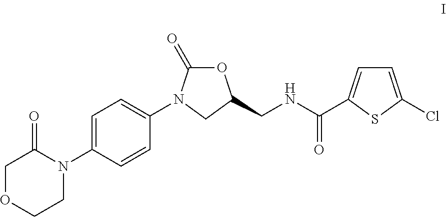

U.S. Pat. No. 7,585,860 describes morpholinyl oxazolidinone thiophene carboxamides including rivaroxaban or pharmaceutically acceptable acid addition salts thereof, a pharmaceutical composition and a method of treatment.

The US'860 patent also discloses a process for the preparation of rivaroxaban which is illustrated by scheme below:

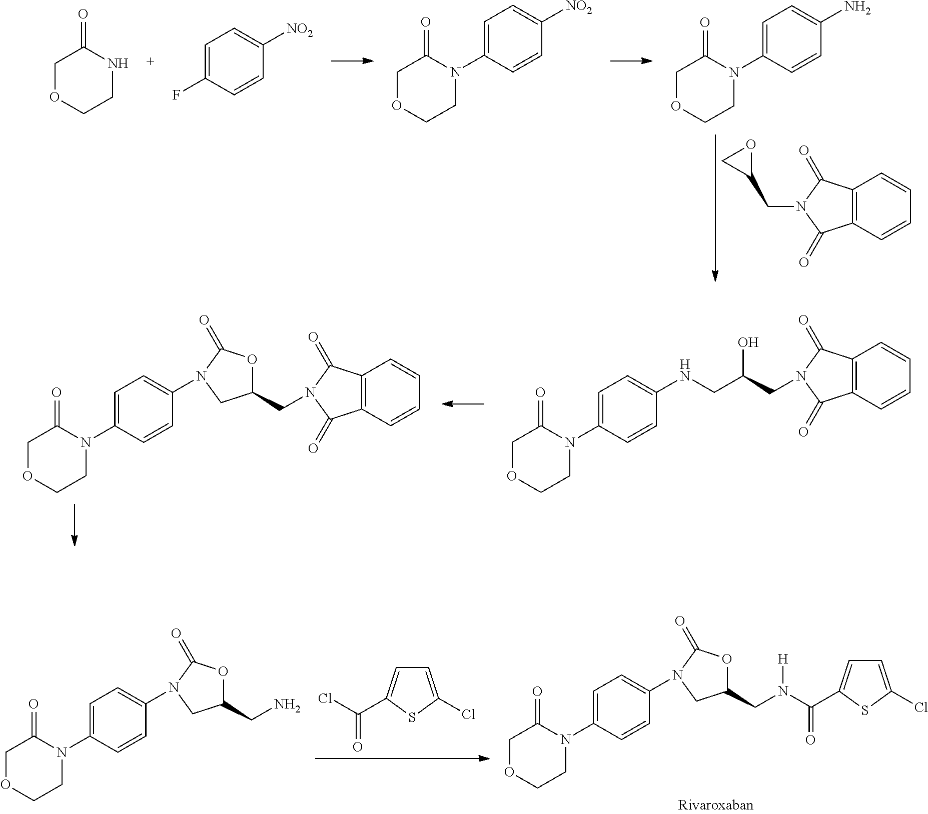

U.S. Publication application US2007/0149522A1 and Drugs of the future 2006, 31(6), 484-493 discloses a process for the preparation of rivaroxaban which is illustrated by scheme below:

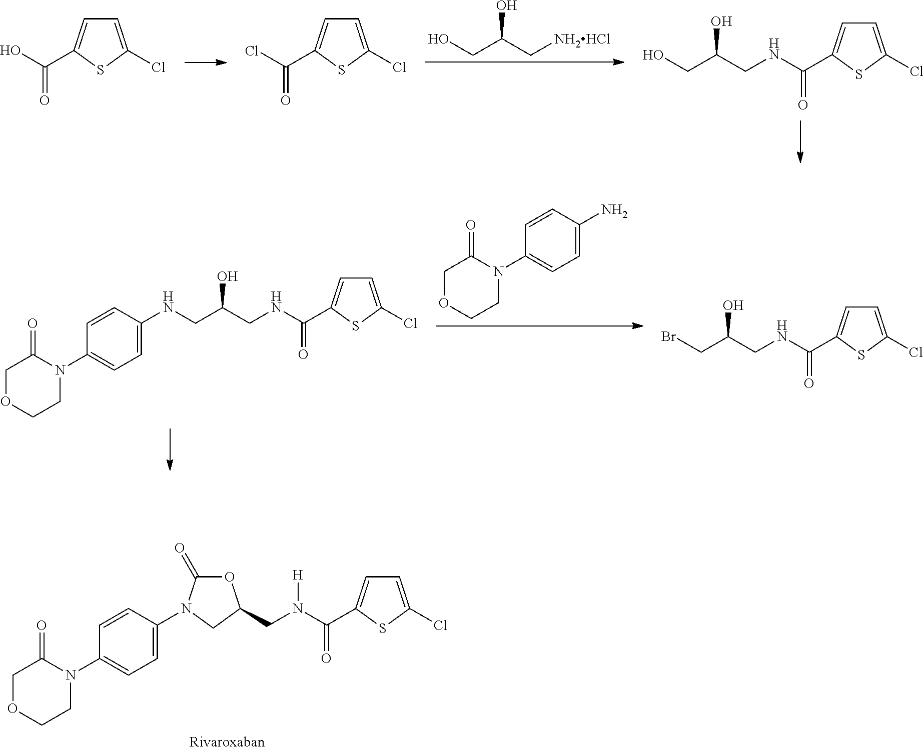

U.S. Pat. No. 7,816,355 B1 describes a process for the preparation of rivaroxaban which is illustrated by below scheme:

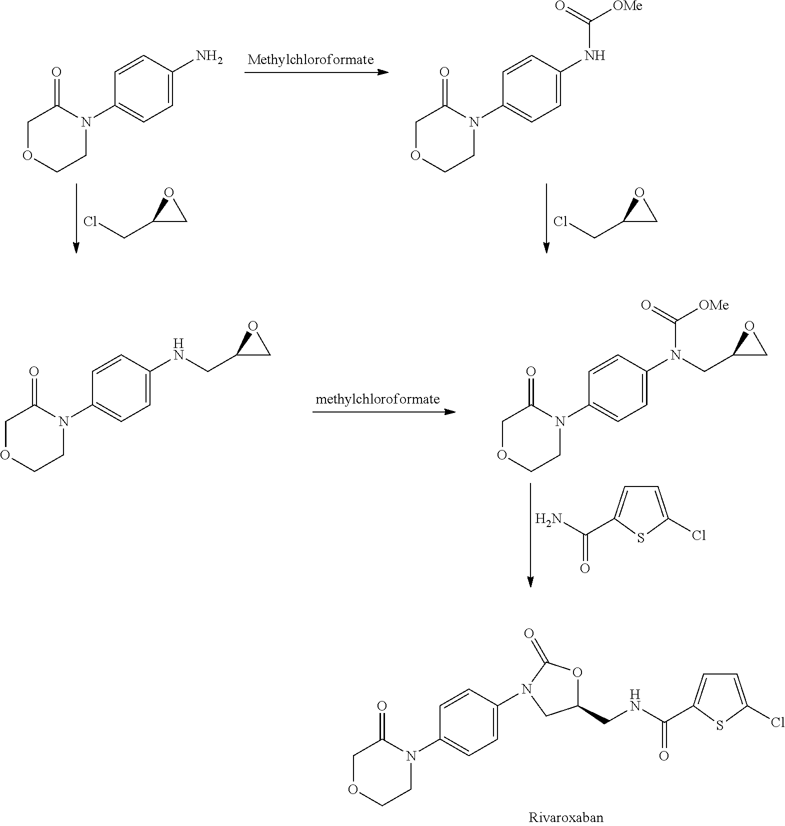

The process disclosed in the US'860 patent exhibits various disadvantages in the reaction management which has particularly unfavourable effects for preparation of the compound of the formula (I) on the industrial scale.

The alternate process disclosed in the U.S. Publication application US'522A1 involves the usage of toxic solvents and reagents. This is disadvantageous per se, and in addition these toxic substances must be removed from the final product (I) until below the maximum limit permissible in each case and may require additional process steps which make the process expensive.

The reported processes aforementioned involves hazardous and expensive reagents like haloformates and bromine derivatives, has more scope for the formation of impurities, intricate to handle on commercial scale, requires additional purification steps thus ending up with low yields and purities of the final product thus rendering the process not amenable on commercial scale.

Keeping the importance of the compound rivaroxaban, there is a need to provide an improved process for the preparation of rivaroxaban, which avoids the use of potentially hazardous, expensive chemicals, the formation of isomeric and other process related impurities, while affording the desired product rivaroxaban in high yield and purity.

The reaction steps of the present invention involving the conversion of compound of formula V to the compound of formula IV followed by conversion of thus obtained compound of formula IV to the compound of formula III of the present invention have not been reported in the literature.

The processes of the present invention are simple, eco-friendly, cost-effective, reproducible, robust and are well amenable on industrial scale.

### SUMMARY OF THE INVENTION

The present invention relates to processes for the preparation of 4-{4-[5(S)-(aminomethyl)-2-oxo-1,3-oxazolidin-3-yl]phenyl}morpholin-3-one of formula (II) a key intermediate in the synthesis of rivaroxaban (I).

In one aspect, the present invention relates to a process for the preparation of compound 4-{4-[5(S)-(aminomethyl)-2-oxo-1,3-oxazolidin-3-yl]phenyl}morpholin-3-one of formula (II) a key intermediate in the synthesis of rivaroxaban (I),

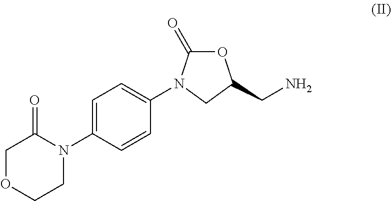

comprising:
a) reacting a compound 4-(4-morpholin-3-onyl)aniline of formula (VII) or a salt thereof

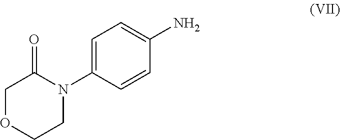 [SMILES: Nc1ccc(N2CCOCC2=O)cc1]

with a compound R-epichlorohydrin of formula (VI)

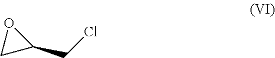

to give the compound 4-[4-(3-chloro-2(R)-hydroxy-propyl amino)-phenyl]-morpholin-3-one of formula (V),

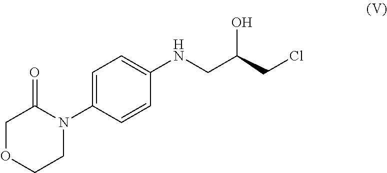 [SMILES: O=C1COCCN1c1ccc(NC[C@@H](O)CCl)cc1]

b) cyclization of the compound of formula (V) or a salt thereof using a suitable reagent to give the compound 4-[4-(5(R)-chloromethyl-2-oxo-oxazolidin-3-yl)-phenyl]-morpholin-3-one of formula (IV)

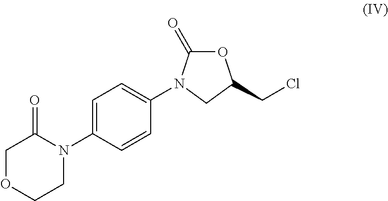

c) reacting the compound of formula (IV) with a suitable reagent to give the compound 2-{2-Oxo-3-[4-(3-oxo-morpholin-4-yl)phenyl]-oxazolidin-5(S)-ylmethyl}-isoindole-1,3-dione of formula (III)

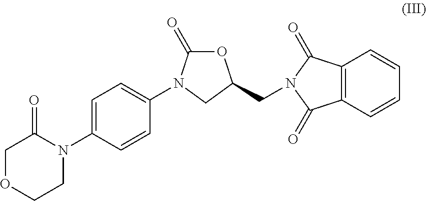 [SMILES: O=C1c2ccccc2C(=O)N1C[C@H]1CN(c2ccc(N3CCOCC3=O)cc2)C(=O)O1]

d) reacting the compound of formula (III) with a suitable reagent to gives the compound of formula (II).

In another aspect, the present invention relates to a process for the preparation of 2-{2-oxo-3-[4-(3-oxo-morpholin-4-yl)-phenyl]-oxazolidin-5(S)-ylmethyl}-isoindole-1,3-dione compound of formula (III)

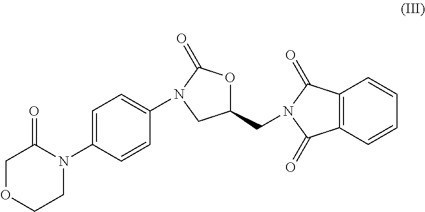 [SMILES: O=C1c2ccccc2C(=O)N1C[C@H]1CN(c2ccc(N3CCOCC3=O)cc2)C(=O)O1]

comprising:
a) reacting the compound 4-[4-(3-Chloro-2(R)-hydroxy-propyl amino)-phenyl]-morpholin-3-one of formula (V) or a salt thereof,

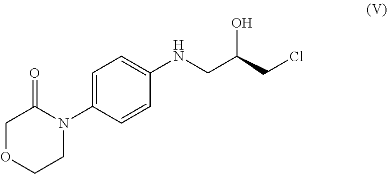 [SMILES: O=C1COCCN1c1ccc(NC[C@@H](O)CCl)cc1]

with a suitable phthalimide derivative to give the 2-((2R)-2-hydroxy)-3-{[4-(3-oxo-4-morpholinyl)-phenyl]amino}-propyl)-1H-isoindole-1,3-(2H)-dione of formula (VIII)

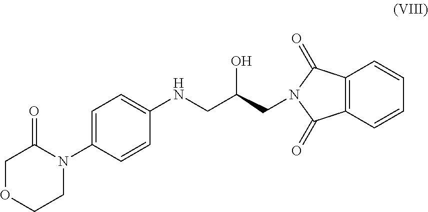 [SMILES: O=C1c2ccccc2C(=O)N1C[C@H](O)CNc1ccc(N2CCOCC2=O)cc1]

b) cyclization of compound of formula (VIII) or a salt thereof using suitable reagent gives the compound of formula (III).

### BRIEF DESCRIPTION OF THE DRAWING

FIG. 1: is a schematic representation of the processes of present invention.

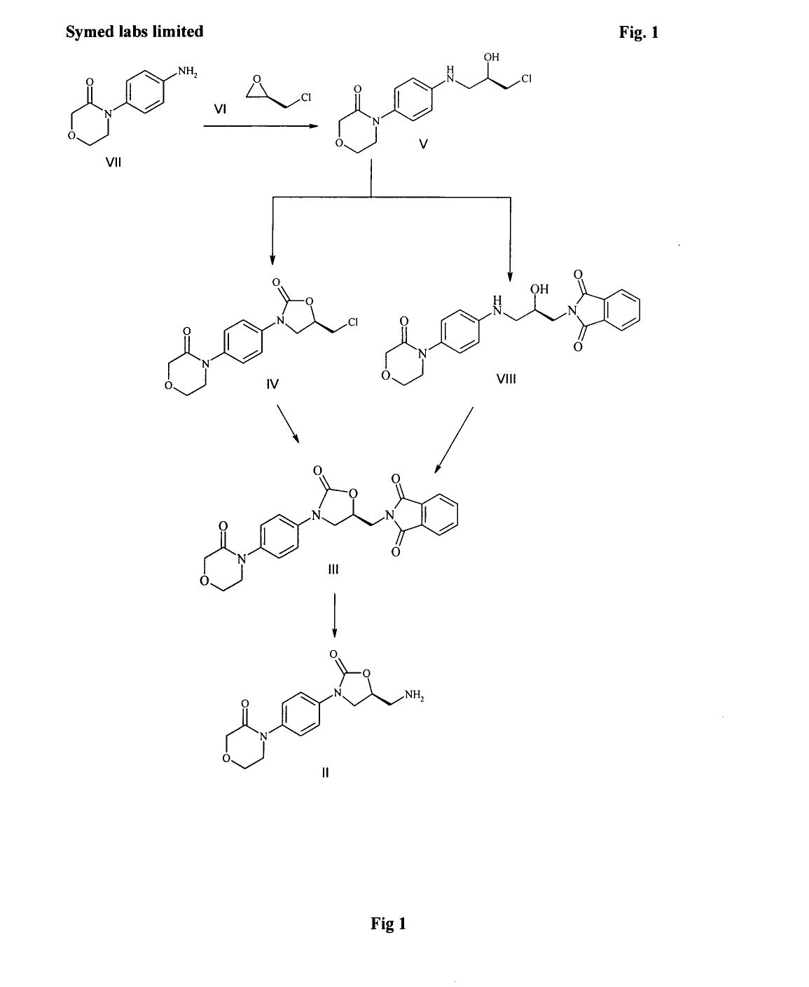

### DETAILED DESCRIPTION OF THE INVENTION

The present invention relates to processes for the preparation of 4-{4-[5(S)-(aminomethyl)-2-oxo-1,3-oxazolidin-3-yl]phenyl}morpholin-3-one of formula (II) a key intermediate in the synthesis of rivaroxaban (I).

In one embodiment, the present invention provides a process for the preparation of compound 4-{4-[5(S)-(aminomethyl)-2-oxo-1,3-oxazolidin-3-yl]phenyl}morpholin-3-one of formula (II) a key intermediate in the synthesis of rivaroxaban (I),

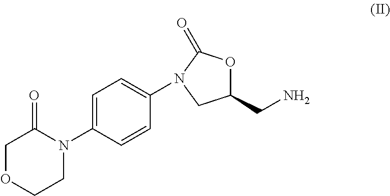 [SMILES: NC[C@H]1CN(c2ccc(N3CCOCC3=O)cc2)C(=O)O1]

comprising:
a) reacting a compound 4-(4-morpholin-3-onyl)aniline of formula (VII) or a salt thereof

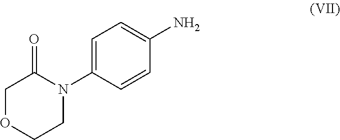 [SMILES: Nc1ccc(N2CCOCC2=O)cc1]

with a compound R-epichlorohydrin of formula (VI)

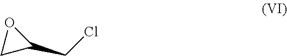

to give the compound 4-[4-(3-chloro-2(R)-hydroxy-propyl amino)-phenyl]-morpholin-3-one of formula (V),

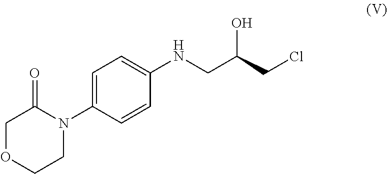 [SMILES: O=C1COCCN1c1ccc(NC[C@@H](O)CCl)cc1]

b) cyclization of the compound of formula (V) or a salt thereof by using a suitable reagent to give the compound 4-[4-(5(R)-chloromethyl-2-oxo-oxazolidin-3-yl)-phenyl]-morpholin-3-one of formula (IV)

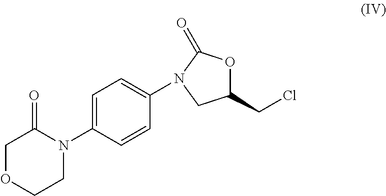

c) reacting the compound of formula (IV) with a suitable reagent to give the compound 2-{2-Oxo-3-[4-(3-oxo-morpholin-4-yl)phenyl]-oxazolidin-5(R)-ylmethyl}-isoindole-1,3-dione of formula (III)

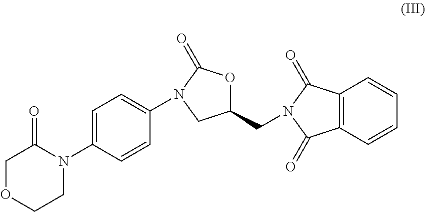 [SMILES: O=C1c2ccccc2C(=O)N1C[C@H]1CN(c2ccc(N3CCOCC3=O)cc2)C(=O)O1]

d) reacting the compound of formula (III) with a suitable reagent to gives the compound of formula (II).

The reaction of step (a) may optionally be carried out in absence of organic solvents. Preferably, the reaction step a) is performed in the presence of organic solvents.

Any solvent, which is neutral towards the reactants are suitable.

The organic solvents that can be used include alcohols such as methanol, ethanol, t-amyl alcohol, t-butyl alcohol and Isopropyl alcohol and the like or aqueous mixtures; cyclic ethers such as tetrahydrofuran and the like; aprotic polar solvents such as N,N-dimethylformamide, N,N-dimethylacetamide, acetonitrile and the like; or mixture thereof. Preferably methanol.

The molar equivalents of compound of formula VI being used can be from about 0.5 to 7.5 moles to the compound of formula VII taken, preferably one mole is being used.

The reaction can be carried out at a temperature range from about 30° C. to about 100° C. or the boiling point of the solvent(s) used, preferably at boiling point of the solvent (s) used.

The time required for the reaction to complete may also vary widely, depending on various factors, notably the reaction temperature, the nature of the reagent and the solvents employed. However, the reaction is effected under the preferred conditions discussed above, a period of from about 1 hour to about 24 hours, preferably from about 5 hour to 16 hours.

The reaction of step (b) is performed using any carbonylating reagent commonly known for such purposes. The carbonylating reagent that can be used include but not limited to carbonyldiimidazole, phosgene, Triphosgene, methyl chloroformate, benzyl chloroformate and phenylchloroformate and the like; carbonyldiimidazole is being preferred.

The molar equivalents of carbonylating reagents being used can be from about 1 to 5 moles to the compound of formula V taken, preferably one mole is being used.

The organic solvents that can be used include but are not limited to halogenated solvents such as dichloromethane, ethylene dichloride, chloroform and the like; esters such as ethyl acetate, isopropyl acetate and the like; hydrocarbon solvents such as n-hexane, cyclohexane, toluene, xylene and the like; ether such as tetrahydrofuran (THF), 1,4-dioxane and the like; aprotic polar solvents such as N,N-dimethylformamide (DMF), dimethylsulfoxide (DMSO), N,N-dimethylacetamide (DMA), N-methylpyrrolidone (NMP) and the like; or mixture thereof in various proportions. Preferably dichloromethane.

The reaction is performed at a temperature range from about 25° C. to about 100° C. or the boiling point of the solvent(s) used, preferably from about 25° C. to about 50° C.

The time required for the reaction to complete may also vary widely, depending on several factors, notably the reaction temperature, the nature of the reagent and solvents employed. The reaction is effected under the preferred conditions at time period from about 1 hour to about 24 hours, preferably from about 10 hours to 20 hours.

The reaction step (c) is a reaction of compound of formula (IV) with a suitable reagent which includes but are not limited to derivatives of phthalimide such as sodium phthalimide, potassium phthalimide and the like; potassium phthalimide is being most preferred.

The molar equivalents of the reagent being used can be from about 1 to 5 moles on the compound of formula IV taken, preferably one mole is being used.

Choosing of solvent is not critical, but preferably the organic solvents must dissolve both the compound of formula VI and reagent making the reaction mixture homogenous and should be neutral, the organic solvents that can be used is selected from the group consisting of halogenated solvents such as dichloromethane, ethylene dichloride, chloroform and the like; esters such as ethyl acetate, isopropyl acetate and the like; aprotic polar solvents such as N,N-dimethylformamide (DMF), dimethylsulfoxide (DMSO), N,N-dimethylacetamide (DMA), N-methylpyrrolidone (NMP) and the like; or mixtures thereof in various proportions. Preferably, N,N-dimethylformamide (DMF) is being used.

The reaction is performed at a temperature range that can be from about 25° C. to about 150° C. or the boiling point of the solvent(s) used, preferably boiling point of the solvent(s) used.

The time required for the reaction to complete may also vary widely, depending on several factors, for example the reaction temperature, the nature of the reagent and solvents employed. However, the reaction is effected at a time period from about 1 hour to about 20 hours, preferably from about 2 hours to about 10 hours.

The reaction step (d) is reaction of the intermediate compound of formula III with suitable reagent in the presence of solvent(s) include but are not limited to hydrazine hydrate or aqueous methyl amine and the like; preferably hydrazine hydrate or aqueous methyl amine.

The organic solvents that can be used is selected from the group consisting of alcohols such as methanol, ethanol, t-amyl alcohol, t-butyl alcohol and Isopropyl alcohol and the like or their aqueous mixtures; cyclic ethers such as tetrahydrofuran and the like; aprotic polar solvents such as N,N-dimethylformamide, N,N-dimethylacetamide, dimethyl sulfoxide, N-methylpyrrolidone (NMP), acetonitrile and the like; or mixture thereof. Preferably methanol.

The reaction temperature can be in the range of about 25° C. to about 150° C. or the boiling point of the solvent(s) used, preferably boiling point of the solvent(s) used.

The time period required for the reaction to complete can be range from about 30 minutes to about 5 hours, preferably 1 hour.

In another embodiment, the present invention provides a process for the preparation of 2-{2-oxo-3-[4-(3-oxo-morpholin-4-yl)-phenyl]-oxazolidin-5(S)-ylmethyl}-isoindole-1,3-dione compound of formula (III)

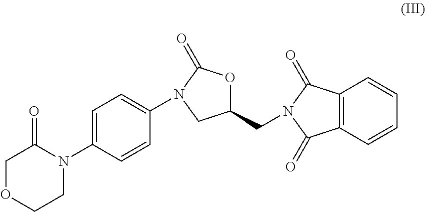 [SMILES: O=C1c2ccccc2C(=O)N1C[C@H]1CN(c2ccc(N3CCOCC3=O)cc2)C(=O)O1]

comprising:
a) reacting the compound 4-[4-(3-Chloro-2(R)-hydroxy-propyl amino)-phenyl]-morpholin-3-one of formula (V) or a salt thereof,

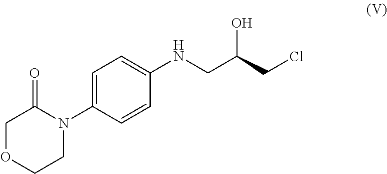 [SMILES: O=C1COCCN1c1ccc(NC[C@@H](O)CCl)cc1]

with a suitable phthalimide derivative to give the 2-((2R)-2-hydroxy)-3-[4-(3-oxo-morpholin-4-yl)-phenylamino]-propyl}-isoindole-1,3-dione of formula (VIII)

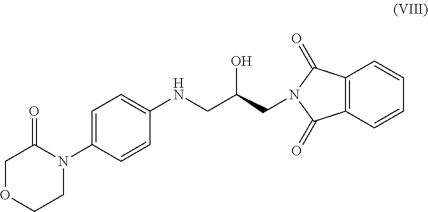 [SMILES: O=C1c2ccccc2C(=O)N1C[C@H](O)CNc1ccc(N2CCOCC2=O)cc1]

b) cyclization of compound of formula (VIII) or a salt thereof using suitable reagent gives the compound of formula (III).

The reaction step (a) is a reaction of compound of formula (V) with a suitable pthalimide derivative which include but are not limited to potassium phthalimide, sodium phthalimide and the like; Potassium phthalimide is being most preferred.

The molar equivalents of reagent being used can be from about 1 to 5 moles on the compound of formula V taken, preferably one mole is being used.

The organic solvents that can be used is selected from the group consisting of halogenated solvents such as dichloromethane, ethylene dichloride, chloroform and the like; esters such as ethyl acetate, isopropyl acetate and the like; aprotic polar solvents such as N,N-dimethylformamide (DMF), dimethylsulfoxide (DMSO), N,N-dimethylacetamide (DMA), N-methylpyrrolidone (NMP) and the like; or mixtures thereof in various proportions. Preferably, N,N-dimethylformamide (DMF) is being used.

Suitably the reaction temperature can be in the range from about 25° C. to about 150° C. or the boiling point of the solvent(s) used, preferably boiling point of the solvent(s) used.

Typically the time required for the reaction to complete may vary depending on factors, like reaction temperature and the nature of the reagent and solvents used. However, the reaction period can be from about 1 hour to about 24 hours, preferably from about 10 hours to 20 hours.

The reaction of step (b) is cyclization of the compound of formula (VIII) can be performed by using any carbonylating reagent commonly known for such purpose. The carbonylating reagent that can be used is selected from the group consisting of carbonyldiimidazole, phosgene, Triphosgene, methyl chloroformate, benzyl chloroformate and phenylchloroformate and the like; carbonyldiimidazole is being more preferred.

The molar equivalents of carbonylating reagents being used can be from about 1 to 5 moles on the compound of formula VIII taken, preferably one mole is being used.

The organic solvents that can be employed in step (b) is selected from the group consisting of halogenated solvents such as dichloromethane, ethylene dichloride, chloroform and the like; esters such as ethyl acetate, isopropyl acetate and the like; hydrocarbon solvents such as n-hexane, cyclohexane, toluene, xylene and the like; ether such as tetrahydrofuran (THF), 1,4-dioxane and the like; aprotic polar solvents such as N,N-dimethylformamide (DMF), dimethylsulfoxide (DMSO), N,N-dimethylacetamide (DMA), N-methylpyrrolidone (NMP) and the like; or mixtures thereof. Preferably dichloromethane.

The reaction is performed at a temperature range from about 25° C. to about 100° C. or the boiling point of the solvent(s) used, preferably from about 25° C. to about 50° C.

The time period for the reaction to complete may vary depending on factors like the temperature, the nature of the reagent and solvent employed. However, the time period is from about 1 hour to about 20 hours, preferably from about 5 hour to 10 hours.

The stereoisomers, for example, can be synthesized by using optically resolved raw material compounds or using a conventional optical resolution or separation method.

It is apparent to one skilled in the art that one could easily perform the identical process steps with the opposite enantiomeric form or racemic form to obtain the corresponding stereoisomers. Therefore, using the chemistry of the claimed process with any of the enantiomeric forms is considered equivalent to the claimed processes.

Optionally the processes for the preparation of intermediates of present invention can be carried out in one pot.

Compound of formula II is a key intermediate in the synthesis of rivaroxaban which are obtained usually in high yields and purity. These compounds may optionally further purified by recrystallization or making slurry in suitable aprotic polar solvent for example acetone, acetonitrile, ethers and or mixtures thereof or by formation of salts for ex. methansulfonate salt. The Examples included in this document illustrate the results obtained regarding purity and yield of these intermediates.

In one embodiment, the intermediates or their salts used here in the processes of the present invention may exist in either crystalline or amorphous or mixtures thereof.

The processes reported for the preparation of intermediates of rivaroxaban results in various process related impurities and bye products thus leading to include additional several purification steps thus resulting in very poor yields and purities of the final product.

The starting intermediate compounds of (VII) and (VI) are commercially available or known per se to the person skilled in the art or can be prepared by processes reported in the literature. For ex. U.S. Pat. No. 7,585,860 which is herein incorporated for reference.

After completion of the reaction, the desired compounds can be obtained from the reaction mixture by conventional means known in the art.

For example, the working-up of reaction mixtures, especially in order to isolate desired compounds, follows customary procedures, known to the organic chemists skilled in the norms of the art and steps, e.g. selected from the group comprising but not limited to extraction, neutralization, crystallization, chromatography, evaporation, drying, filtration, centrifugation and the like.

Optionally the process steps of present invention can be carried out by one pot synthesis independently.

The reported processes aforementioned involves hazardous and expensive reagents like haloformates and bromine derivatives has more scope for the formation of impurities, difficult to handle on commercial scale and also requires additional purification steps thus ending up with low yields and purities of the final product thus rendering the process not amenable on commercial scale.

The processes of present invention are especially valuable for the following reasons: it makes it possible to obtain the intermediate compounds on an industrial scale in excellent yields, starting from a simple, low-cost starting materials, involve simple process steps and reagents thus making processes more cost effective than reported processes.

Advantageously, the processes of present invention do not involve purification steps thus provides the intermediates of rivaroxaban with higher yields and purities.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

### Example 1

### Preparation of 4-[4-(3-chloro-2(R)-hydroxypropyl amino)-phenyl]-morpholin-3-one (V)

4-(4-Morpholin-3-onyl)aniline (39 g), R-epichlorohydrin (18.5 g) and methanol (200 ml) were charged into a clean and dry 4 neck R.B.flask followed by heating to about reflux for about 16 hours. After completion of the reaction, the solvent was distilled completely to give 57 gms of the title compound.

### Example 2

### Preparation of 4-[4-(5(R)-chloromethyl-2-oxo-oxazolidin-3-yl)-phenyl]-morpholin-3-one (IV)

57 gms of 4-[4-(3-chloro-2-hydroxy-propyl amino)-phenyl]-morpholin-3-one and methylene chloride (600 ml) were charged into a clean and dry 4 neck R.B.flask. 32 gms of carbonyl diimidazole was added at about 30° C. and the resultant reaction mixture was stirred for about 20 hours. After completion of the reaction, reaction mixture was washed with water and methylene chloride was distilled completely to give 48 gms of the title compound.

### Example 3

### Preparation of 2-5(S){2-oxo-3-[4-(3-oxo-morpholin-4-yl)-phenyl]-oxazolidin-5-ylmethyl}-isoindole-1,3-dione (III)

60 gms of 4-[4-(5-chloromethyl-2-oxo-oxazolidin-3-yl)-phenyl]-morpholin-3-one, potassium phthalimide (40 g) and N,N-dimethyl formamide (400 ml) were charged into a clean and dry 4 neck R.B.flask. The resultant reaction mixture was heated to reflux for about 5 hours. After completion of the reaction, the reaction mixture was cooled to about 30° C., poured into 2 L of water and the solid separated was filtered to give 50 gms of the title compound.

### Purification of Intermediate Compound of Formula III Using DMF and Acetone

50 g of crude compound of formula III and 125 ml DMF were charged into a clean and dry 4 neck R.B.flask and heated to about 90° C., the clear solution obtained, carbon (5 g) was charged. The reaction suspension was stirred for 5 mins and filtered under hot conditions. The filtrate was cooled to about 30° C., 150 ml of acetone was added and the solid separated was filtered after 30-45 min and washed with acetone (50 ml) to afford 42.5 g of pure product as half white colored solid.

### Purification of Intermediate Compound of Formula III Using DMF and Methanol

Dissolve 50 g of crude compound in 235 ml DMF at 90-95° C., to the clear solution add carbon (5 g), filter after 5 min under hot conditions. Cool the filterate to 25-30° C., add 125 ml of methanol and filter the solid after 30-45 min and wash with methanol (50 ml) to yield 40 g of pure product of off white colored solid.

### Example 4

### Preparation of 4-{4-[5(S)-(amino methyl)-2-oxo-1,3-oxazolidin-3-yl]phenyl}morpholin-3-one (II)

Methanol (240 ml) and Hydrazine hydrate (26 g) were added to a flask containing the (2-{2-Oxo-3-[4-(3-oxo-morpholin-4-yl)-phenyl]-oxazolidin-5-ylmethyl}-isoindole-1,3-dione (40 g), heated for 1 hour at reflux temperature and cooled to room temperature. After completion of the reaction, 500 ml of water was added to the reaction mass and was extracted with methylene dichloride (300 ml). The combined extractions were washed with water (100 ml) and the solvent was distilled completely to give 20 gms of the title compound.

### Example-5

### Preparation of 2-{2(R)-Hydroxy-3-[4-(3-oxo-morpholin-4-yl)phenyl amino]-propyl}-isoindole-1,3-dione (VIII)

(50 g) 4-[4-(3-Chloro-2-hydroxy-propylamino)-phenyl]-morpholin-3-one, (45 g) of potassium phthalimide and (100 ml) N,N-dimethyl formamide (DMF) were charged into a clean and dry 4 neck R.B.flask. The resultan reaction mixture was heated to reflux for about 5 hours After completion of the reaction, the reaction mixture was cooled to about 30° C. and quenched with 2 L water and the solid separated was filtered to give 60 gms of title compound.

### Example-6

### Preparation of 2-5(S)-{2-Oxo-3-[4-(3-oxo-morpholin-4-yl)phenyl]-oxazolidin-5-ylmethyl}-isoindole-1,3-dione (III)

60 gms of 2-{2-Hydroxy-3-[4-(3-oxo-morpholin-4-yl)phenylamino]-propyl}-isoindole-1,3-dione and 180 ml of methylene chloride were charged into a clean and dry 4 neck R.B.flask. 29 gms of carbonyl diimidazole was added at about 30° C. and the resultant reaction mixture was stirred at about 30° C. for about 20 hours. After completion of the reaction, the reaction mixture was washed with water and the solvent was distilled completely to give 55 gms of the title compound.

### Example-7

### Preparation of 2-5(S)-{2-Oxo-3-[4-(3-oxo-morpholin-4-yl)phenyl]-oxazolidin-5-ylmethyl}-isoindole-1,3-dione (III) by one pot process

A mixture of (50 g) 4-[4-(3-Chloro-2-hydroxy-propylamino)-phenyl]-morpholin-3-one and (45 g) isoindole-1,3-dione and (100 ml) N,N-dimethyl formamide (DMF) was heated to reflux temperature for about 5 hours. After completion of the reaction, the reaction mixture was cooled to about 30° C. and quenched with 2 L water and the solid separated was filtered to give 60 gms of -{2-Hydroxy-3-[4-(3-oxo-morpholin-4-yl)phenylamino]-propyl}-isoindole-1,3-dione crude. To this 180 ml) of methylene dichloride and 29 gms of carbonyl diimidazole was added at about 30° C. and the reaction mixture was stirred for about 20 hours. After completion of the reaction, the reaction mixture was washed with water and the solvent was distilled completely to give 55 gms of the title compound.

### Example-8

### Preparation of 4-[4-(5(R)-chloromethyl-2-oxo-oxazolidin-3-yl)-phenyl]-morpholin-3-one (IV) using triphosgene

4-[4-(3-chloro-2-hydroxy-propyl amino)-phenyl]-morpholin-3-one (57 g) and chloroform (600 ml) were charged into a clean and dry 4 neck R.B.flask. triphosgene (32 g) was added at about 30° C. and the resultant reaction mixture was stirred at about 30° C. for about 20 hours. After completion of the reaction, reaction mixture was washed with water and chloroform was distilled completely to give 48 gms of the title compound.

1-29. (canceled)

30. A process for the preparation of 4-{4-[5(S)-(aminomethyl)-2-oxo-1,3-oxazolidin-3-yl]phenyl}morpholin-3-one of the formula (II), 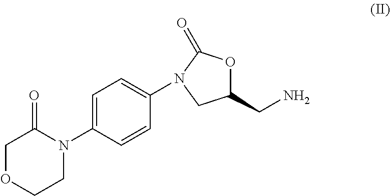
comprising:
a) reacting 4-(4-morpholin-3-onyl)aniline of the formula (VII) or a salt thereof 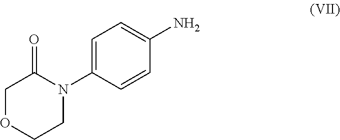 [SMILES: Nc1ccc(N2CCOCC2=O)cc1]
with R-epichlorohydrin of the formula (VI), 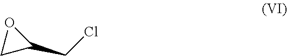
in an organic solvent selected from the group consisting of an alcohol selected from the group consisting of methanol, ethanol, isopropanol, t-amyl alcohol, t-butyl alcohol, hexanol, and octanol, ethers selected from the group consisting of tetrahydrofuran and 1,4-dioxane and aprotic solvents selected from the group consisting of acetonitrile, N,N-dimethyl formamide (DMF), N,N-dimethyl acetamide, dimethyl sulfoxide (DMSO), N-methylpyrrolidone (NMP) or mixtures thereof, to give 4-[4-(3-chloro-2(R)-hydroxy-propyl amino)-phenyl]-morpholin-3-one of the formula (V), 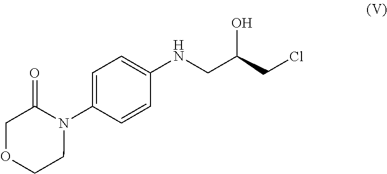 [SMILES: O=C1COCCN1c1ccc(NC[C@@H](O)CCl)cc1]
b) cyclization of the formula (V) compound or a salt thereof using a suitable reagent selected from the group consisting of carbonyl diimidazole, phosgene, triphosgene, methyl chloroformate, benzyl chloroformate and phenyl chloroformate in the presence of an organic solvent selected from the group consisting of halogenated solvents selected from the group consisting of dichloromethane, ethylene dichloride and chloroform, esters selected from the group consisting of ethyl acetate and isopropyl acetate, hydrocarbon solvents selected from toluene and xylene, ethers selected from tetrahydrofuran, 1,4-dioxane, aprotic polar solvents selected from the group consisting of acetonitrile, N,N-dimethyl formamide (DMF), dimethyl sulfoxide (DMSO), N,N-dimethyl acetamide and N-methylpyrrolidone (NMP) or mixtures thereof to give 4-[4-(5(R)-chloromethyl-2-oxo-oxazolidin-3-yl)-phenyl]-morpholin-3-one of the formula (IV) 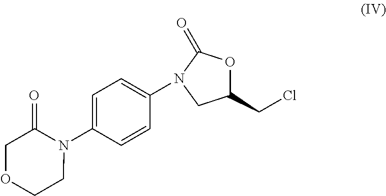
c) reacting the formula (IV) compound with potassium phthalimide in the presence of an organic solvent selected from the group consisting of halogenated solvents selected from the group consisting of dichloromethane, ethylene dichloride and chloroform, esters selected from the group consisting of ethyl acetate and isopropyl acetate, aprotic polar solvents selected from the group consisting of acetonitrile, N,N-dimethyl formamide (DMF), dimethyl sulfoxide (DMSO), N,N-dimethyl acetamide and N-methylpyrrolidone (NMP) or mixtures thereof to give 2-{2-oxo-3-[4-(3-oxo-morpholin-4-yl)phenyl]-oxazolidin-5(S)ylmethyl}-isoindole-1,3-dione of the formula (III), and 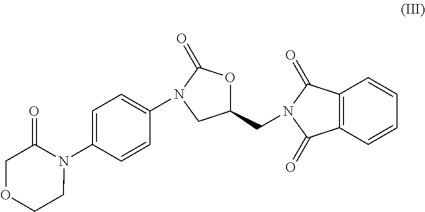 [SMILES: O=C1c2ccccc2C(=O)N1C[C@H]1CN(c2ccc(N3CCOCC3=O)cc2)C(=O)O1]
d) reacting the formula (III) compound with a suitable reagent selected from the group consisting of hydrazine hydrate and aqueous methyl amine in the presence of an organic solvent selected from the group consisting of alcohols selected from the group consisting of methanol, ethanol, isopropanol, t-amyl alcohol, t-butyl alcohol, hexanol and octanol, ethers selected from the group consisting of tetrahydrofuran and 1,4-dioxane, aprotic polar solvents selected from the group consisting of acetonitrile, N,N-dimethyl formamide (DMF), dimethyl sulfoxide (DMSO), N,N-dimethyl acetamide and N-methylpyrrolidone (NMP) or mixtures thereof or their aqueous mixtures to give the formula II compound.

31. The process of claim 30, wherein the reaction step (a) is performed at a temperature range of about 25° C. to about 100° C. or the boiling point of the solvent(s) used, wherein the time required for the reaction step (a) for completion is from about 1 hour to about 24 hours.

32. The process of claim 30, wherein the reaction step (b) is performed at a temperature range of about 25° C. to about 100° C. or the boiling point of the solvent(s) used, wherein the time required for the reaction step (b) for completion is from about 1 hour to about 24 hours.

33. The process of claim 30, wherein the reaction step (c) is performed at a temperature range of about 25° C. to about 150° C. or the boiling point of the solvent(s) used, wherein the time required for the reaction step (b) for completion is from about 1 hour to about 20 hours.

34. The process of claim 30, wherein the reaction step (d) is carried out at temperature from about 25° C. to about 150° C. or the boiling point of the solvent(s) used, wherein the time required for the reaction step (d) to complete is from about 30 minutes to about 5 hours.

35. A process for the preparation of 4-[4-(5(R)-chloromethyl-2-oxo-oxazolidin-3-yl)-phenyl]-morpholin-3-one of the formula (IV) 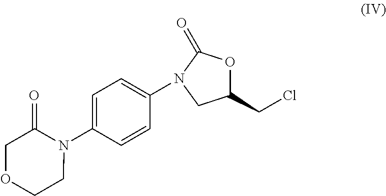
by cyclization of 4-[4-(3-chloro-2(R)-hydroxy-propyl amino)-phenyl]-morpholin-3-one of the formula (V) or a salt thereof 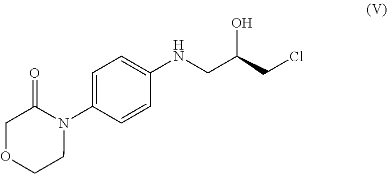 [SMILES: O=C1COCCN1c1ccc(NC[C@@H](O)CCl)cc1]
using a suitable reagent selected from the group consisting of carbonyl diimidazole, phosgene, triphosgene, methyl chloroformate, benzyl chloroformate and phenyl chloroformate.

36. A process for the preparation of 2-{2-oxo-3-[4-(3-oxo-morpholin-4-yl) phenyl]oxazolidin-5(S)ylmethyl}-isoindole-1,3-dione of the formula (III) 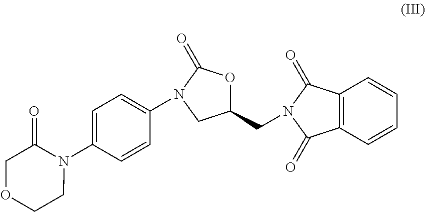 [SMILES: O=C1c2ccccc2C(=O)N1C[C@H]1CN(c2ccc(N3CCOCC3=O)cc2)C(=O)O1]
by reacting 4-[4-(5(R)-chloromethyl-2-oxo-oxazolidin-3-yl)-phenyl]-morpholin-3-one of the formula (IV) 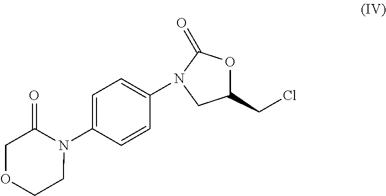
with potassium phthalmide.

37. A process for the preparation of 4-{4-[5(S)-(aminomethyl)-2-oxo-1,3-oxazolidin-3-yl]phenyl}morpholin-3-one of the formula (II), 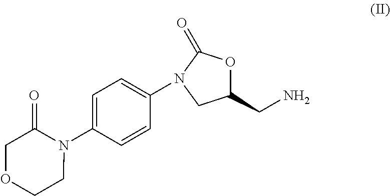
by reacting 2-{2-oxo-3-[4-(3-oxo-morpholin-4-yl)phenyl]-oxazolidin-5(S)ylmethyl}-isoindole-1,3-dione of the formula (III) 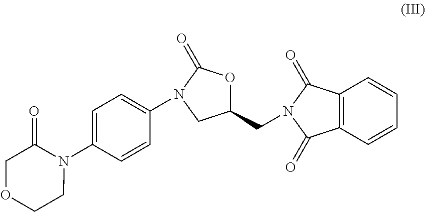 [SMILES: O=C1c2ccccc2C(=O)N1C[C@H]1CN(c2ccc(N3CCOCC3=O)cc2)C(=O)O1]
using hydrazine hydrate.

38. A process for the preparation of 2-{2-Oxo-3-[4-(3-oxo-morpholin-4-yl)-phenyl]-oxazolidin-5(S)-ylmethyl}-isoindole-1,3-dione of the formula (III) 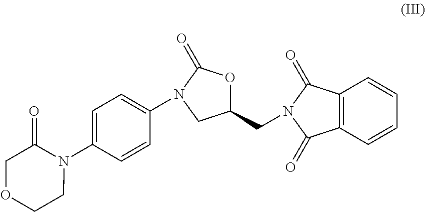 [SMILES: O=C1c2ccccc2C(=O)N1C[C@H]1CN(c2ccc(N3CCOCC3=O)cc2)C(=O)O1]
comprising:
a) reacting 4-[4-(3(R)-Chloro-2-hydroxy-propyl amino)-phenyl]-morpholin-3-one of the formula (V) or a salt thereof, 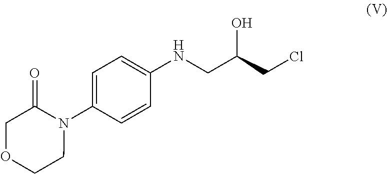 [SMILES: O=C1COCCN1c1ccc(NC[C@@H](O)CCl)cc1]
with a suitable phthalimide derivative selected from the group consisting of sodium phthalimide, potassium phthalimide or mixtures thereof in the presence of an organic solvent selected from the group consisting of halogenated solvents selected from the group consisting of dichloromethane, ethylene dichloride and chloroform, esters selected from the group consisting of ethyl acetate and isopropyl acetate and aprotic polar solvents selected from the group consisting of acetonitrile, N,N-dimethyl formamide (DMF), dimethyl sulfoxide (DMSO), N,N-dimethyl acetamide and N-methylpyrrolidone (NMP) or mixtures thereof,
to give 2-{2(R)-hydroxy-3-[4-(3-oxo-morpholin-4-yl)-phenylamino]-propyl}-isoindole-1,3-dione of the formula (VIII), and 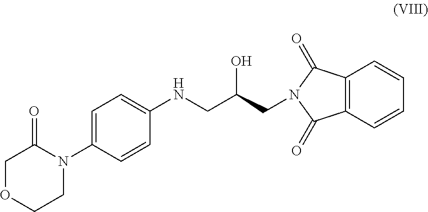 [SMILES: O=C1c2ccccc2C(=O)N1C[C@H](O)CNc1ccc(N2CCOCC2=O)cc1]
b) cyclization of the formula (VIII) compound or a salt thereof using a suitable reagent selected from the group consisting of carbonyldiimidazole, phosgene, triphosgene, methyl chloroformate, benzyl chloroformate and phenylchloroformate or mixtures thereof in the presence of an organic solvent selected from halogenated solvents selected from dichloromethane, ethylene dichloride and chloroform, esters selected from the group consisting of ethyl acetate and isopropyl acetate, hydrocarbon solvents selected from the group consisting of toluene and xylene, ethers selected from the group consisting of tetrahydrofuran, 1,4-dioxane aprotic polar solvents selected from the group consisting of acetonitrile, N,N-dimethyl formamide (DMF), dimethyl sulfoxide (DMSO), N,N-dimethyl acetamide and N-methylpyrrolidone (NMP) or mixtures thereof to give a formula (III) compound.

39. The process of claim 38, wherein the reaction temperature in step (a) is from about 25° C. to about 150° C. or the boiling point of the solvent(s) used, wherein the time required in reaction step (a) is from about 1 hour to about 20 hours, preferably from about 5 hour to 10 hours.

40. The process of claim 38, wherein reaction step (b) is performed at a temperature range from about 25° C. to about 100° C. or the boiling point of the solvent(s) used, wherein the time period required in reaction step (b) is from about 1 hour to about 24 hours.

41. A process for the preparation of 4-[4-(3-chloro-2(R)-hydroxy-propyl amino)-phenyl]-morpholin-3-one of the formula (V), 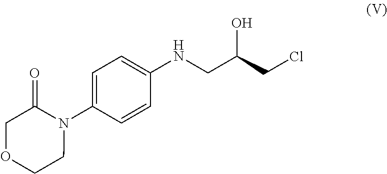 [SMILES: O=C1COCCN1c1ccc(NC[C@@H](O)CCl)cc1]
by reacting 4-(4-morpholin-3-onyl)aniline of the formula (VII) or a salt thereof 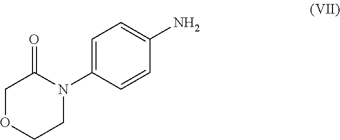 [SMILES: Nc1ccc(N2CCOCC2=O)cc1]
with R-epichlorohydrin of the formula (VI) 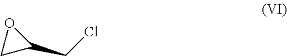
to give the formula V compound.

42. Use of a formula II compound prepared according to claim 30 in the synthesis of a formula I oxazolidine morpholinone derivative rivaroxaban compound.
